(19) United States
(12) Patent Application Publication
(10) Pub. No.: US 2023/0145965 A1
(43) Pub. Date: May 11, 2023

(54) TISSUE DEBULKING DEVICE

(71) Applicant: CAREVATURE MEDICAL LTD., Rehovot (IL)

(72) Inventors: David SKOROHOD, Netanya (IL); Eran MILLER, Moshav Beit Elazari (IL); Yoseph WEITZMAN, Tel Aviv (IL)

(21) Appl. No.: 17/918,362

(22) PCT Filed: Apr. 12, 2021
(86) PCT No.: PCT/IL2021/050416

§ 371 Date: Oct. 12, 2022

Related U.S. Application Data
(60) Provisional application No. 63/010,959, filed on Apr. 16, 2020.

(51) Int. Cl. A61B 17/16

(52) U.S. Cl. CPC A61B 17/1604; A61B 17/1631; A61B 17/1628; A61B 17/1633; A61B 2090/034

(57) ABSTRACT

Disclosed herein is a surgical tool for debulking hard tissue. The surgical tool includes: (i) an elongated hollow member including a distally located bent section; (ii) a cable extending within the hollow member, along a predetermined length thereof; (iii) a headpiece positioned at, or distally to, the bent section; (iv) a rotation actuator coupled to the cable proximal end and configured to rotate the cable about a longitudinal axis thereof; and (v) a motion converter coupled to a distal end of the cable and to the headpiece, at least part of the motion converter is positioned in, and/or distally, to the bent section, the motion converter being configured to transform rotational motion of the cable into an axial, reciprocating motion of the headpiece. The headpiece is configured to break up hard tissue by hammering thereof, when effecting axial, reciprocating motion, while simultaneously minimizing damage to soft tissue if struck.

### TECHNICAL FIELD

The present disclosure relates generally to surgical tools, and related methods, for tissue removal.

### BACKGROUND

Excess body tissue can lead to pathological conditions giving rise to pain, especially when the excess body tissue impinges on a nerve. One such common condition is spinal stenosis: narrowing (stenosis) of the spinal canal, due to excess bone tissue pressing on the spinal cord and resulting in a neurological deficit. Other such common conditions include bulging or herniated discs, which are associated with osteophyte formation in the spinal canal.

Standard treatments for spinal stenosis include corpectomy, laminectomy, and osteotomy: surgical procedures involving removing from a vertebra any bone spurs pressing on the spinal cord, and thereby decompressing the spinal cord and nerves and alleviating the neurological deficit. Treatment of a herniated disc typically involves a surgical procedure called discectomy, during which herniated disc material that presses against the nerve root or spinal cord is removed.

These surgical procedures, and others, require selective removal of target tissue—which is often difficult to reach—while avoiding damage to surrounding tissue. This task is made doubly difficult when, in addition, the target tissue is hard tissue, such as resulting from excess bone growth on a vertebra.

### SUMMARY

Aspects of the disclosure, according to some embodiments thereof, relate to surgical tools, and related methods, for tissue removal. More specifically, but not exclusively, aspects of the disclosure, according to some embodiments thereof, relate to surgical tools configured for breaking up hard/hardened tissue by hammering/pounding/grounding/grating the tissue.

The surgical tools and methods of the present disclosure allow for selective removal of tissue in difficult-to-reach anatomical sites while minimizing damage to surrounding tissue. Advantageously, the surgical tools and methods of the present disclosure make use of the mechanical properties of the tissue, that is to be removed, in order to prevent/mitigate damage to surrounding tissue. According to some embodiments, the surgical tools and methods of the present disclosure are configured for hammering tissue, and, as such, are adapted to debulk hard tissue (such as bone), which is breakable/brittle, while avoiding/mitigating damage to soft tissue (around the bone), which is elastic. More specifically, by virtue of its rigidity, bone poses resistance to hammering by a hard tool, and, as such, is debulked thereby. In contrast, by virtue of its elasticity, soft tissue is not harmed, or substantially not harmed, by hammering by a hard and blunt tool, as the soft tissue moves back and forth with the tool. Advantageously, the inherent safety exhibited by the surgical tools and methods of the present disclosure with respect to soft tissue, may allow to shorten the duration of hard tissue removal procedures, which are performed in close vicinity to neural elements.

Thus, according to an aspect of some embodiments, there is provided a surgical tool for debulking hard tissue. The surgical tool includes:
A hollow member, which is elongated and includes a distally located bent section.
A cable extending within the hollow member, along a predetermined length thereof, from a cable proximal end to a cable distal end. The cable is configured to resist helixing.
A headpiece positioned at, or distally to, the bent section.
A rotation actuator coupled to the cable proximal end and configured to rotate the cable about a longitudinal axis thereof.
A motion converter coupled to the cable distal end and to the headpiece. At least a part of the motion converter is positioned in, and/or distally, to the bent section. The motion converter is configured to transform rotational motion of the cable into an axial, reciprocating motion of the headpiece.

The headpiece is configured to break up hard tissue by hammering thereof, when effecting axial, reciprocating motion, while simultaneously minimizing damage to soft tissue if struck.

According to some embodiments, the surgical tool is configured to allow the headpiece to strike hard tissue at rate of at least about 10,000 strikes per minute (SPM).

According to some embodiments, the motion converter includes a cam, coupled to the cable distal end, and a pushrod mechanically coupled to, or including, the headpiece. The pushrod is configured to engage the cam and to effect axial, reciprocating motion.

According to some embodiments, the surgical tool further includes a main section and a distal section. The bent section is joined on a proximal end thereof to the main section and on a distal end thereof to the distal section. The cam is positioned in the distal section.

According to some embodiments, at least during a part of the axial, reciprocating motion, the headpiece distally projects from a distal end of the distal section.

According to some embodiments, at least a portion of the cable, which extends along the bent section, is flexible.

According to some embodiments, the surgical tool further includes a stopper mechanism configured to restrict axial displacement of the pushrod in at least the distal direction.

According to some embodiments, the pushrod includes a pinhole extending from one side-surface thereof to an opposite side-surface thereof The stopper mechanism includes a pin extending through the pinhole from one sidewall of the distal section to an opposite sidewall thereof The pinhole may have a diameter greater than the pin, thereby restricting axial displacement of the pushrod while allowing for axial reciprocating motion of the pushrod.

According to some embodiments, the surgical tool further includes a linear-motion bearing, positioned at the distal end of the distal section such that the pushrod extends therethrough. The linear-motion bearing is configured to facilitate axial, reciprocating motion of the pushrod.

According to some embodiments, the motion converter includes a sleeve element mountable on or insertable into the distal section. The sleeve element includes the pushrod which is at least partially disposed within and along the sleeve element.

According to some embodiments, the pushrod includes a pinhole extending from one side-surface thereof to an opposite side-surface thereof The stopper mechanism includes a pin extending through the pinhole from one sidewall of the sleeve element to an opposite sidewall thereof The pinhole may have a diameter greater than the pin, thereby restricting axial displacement of the pushrod while allowing for axial reciprocating motion of the pushrod.

According to some embodiments, the cam includes one or more lobes, projecting from a distal end of the cam, such as to engage a proximal end of the pushrod when the cam effects rotary motion.

According to some embodiments, a distal end of the cam defines an oblique surface, the cam being thereby configured to engage a proximal end of the pushrod when the cam effects rotary motion.

According to some embodiments, the pushrod extends in parallel, or substantially in parallel, to a central axis of the distal section and is displaced relative to the central (longitudinal) axis of the distal section.

According to some embodiments, the surgical tool is configured to effect the axial, reciprocating motion of the headpiece at rate of at least about 10,000 SPM when the angle is smaller than 180° and the cable is bent at a bend radius below 10 mm.

According to some embodiments, the surgical tool is further configured to operate such that the cable rotates at a rate of at least about 10,000 revolutions per minute (RPM).

According to some embodiments, the surgical tool is further configured to effect axial, reciprocating motion such as to allow hammering a rate of up to about 240,000 SPM.

According to some embodiments, the cable includes a plurality of braided/intertwined/stranded wires.

According to some embodiments, the hard tissue is bone tissue.

According to some embodiments, the surgical tool further includes a handle configured to facilitate operation and control of the surgical tool by an operator.

According to some embodiments, a distal tip of the headpiece includes an eroding surface, including one or more protrusions, and configured for hammering hard tissue.

According to some embodiments, a circumferential surface of the headpiece is eroding, the surgical tool being thereby further configured for debulking hard tissue by grating.

According to an aspect of some embodiments, there is provided a surgical tool for debulking hard tissue. The surgical tool includes:
A hollow member, which is elongated and includes a main section and a distal section. The distal section is positioned at an angle relative to the main section.
A cable, which is elongated and includes a cable proximal end and a cable distal end. The cable extends within the main section there along and is configured to resist helixing.
A work element exposed on a sidewall of the distal section. The work element is configured for transverse, reciprocating motion, wherein, at least during a part of the transverse, reciprocating motion, the work element radially projects from the sidewall.
A rotation actuator coupled to the cable proximal end and configured to rotate the cable about a longitudinal axis thereof.
A motion converter coupled to the cable distal end and to the work element. The motion converter is configured to transform rotational motion of the cable into the transverse, reciprocating motion of the work element.

The work element includes a radially facing eroding surface. The work element is configured to break up hard tissue by hammering thereof, when effecting transverse, reciprocating motion, while simultaneously minimizing damage to soft tissue if struck.

According to some embodiments, the motion converter includes a rotatable cam and a resilient member. The work element is coupled to the resilient member. The cam includes one or more projections configured to laterally push the work element as the cam revolves. The resilient member is configured to exert a return force on the work element when the work element projects from the sidewall. The work element is thereby configured to effect the transverse, reciprocating motion when the cam revolves.

According to some embodiments, the resilient member is a spring.

According to some embodiments, the spring is a leaf spring.

According to some embodiments, the one or more projections are configured to directly laterally push the work element as the cam revolves.

According to an aspect of some embodiments, there is provided a surgical tool for debulking hard tissue. The surgical tool includes:
An elongated hollow member including a main section and a distal section.
A cable extending within the hollow member along a predetermined length thereof, from a cable proximal end to a cable distal end. The cable is configured to resist helixing.
A headpiece positioned at the distal section.
A rotation actuator coupled to the cable proximal end and configured to rotate the cable about a longitudinal axis thereof.
A motion converter positioned in, or in proximity to, the distal section. The motion converter is coupled to the cable distal end and to the headpiece. The motion converter is configured to transform rotation of the cable into an axial or transverse, reciprocating motion of the headpiece.

The headpiece is configured to break up hard tissue by hammering thereof, when effecting axial or transverse, reciprocating motion, while simultaneously minimizing damage to soft tissue if struck.

According to some embodiments, the distal section is set at an angle relative to the main section.

According to some embodiments, at least a portion of the cable, which extends along a bent section joining the main section to the distal section, is flexible.

According to some embodiments, the headpiece is positioned at the distal section of the hollow member, such as to be excentric.

According to some embodiments, the surgical tool further includes one or more electrodes positioned on a distal tip of hollow member, such as to render the surgical tool configured for electrophysiological monitoring and/or neurostimulation.

According to some embodiments, the surgical tool further includes one or more electrical wires embedded within a wall of the hollow member such that respective distal ends of the one or more wires are connected to the one or more electrodes respectively.

According to some embodiments, at least part of the hollow member is made of an electrically conducting material(s) extending along a length of the hollow member until the distal tip thereof The distal tip constitutes the one or more electrodes and is configured to function as a single electrode. The hollow member is thereby configured to allow establishing a voltage between the one or more electrodes and an external electrode placed on/in a body of a subject during a hard tissue debulking procedure.

According to some embodiments, at least part of the hollow member is made of an electrically conducting material(s) extending along a length of the hollow member until the distal tip thereof The one or more electrodes include at least two electrodes. The distal tip constitutes the at least two electrodes and is configured to function as two electrodes.

The hollow member is thereby configured to allow establishing a voltage difference between the at least two electrodes.

According to some embodiments, the surgical tool is configured to allow the headpiece to strike hard tissue at rate of at least about 10,000 strikes per minute (SPM).

According to some embodiments, the motion converter includes a cam, coupled to the cable distal end, and a pushrod mechanically coupled to, or including, the headpiece. The pushrod is configured to engage the cam and to effect axial, reciprocating motion.

According to some embodiments, at least during a part of the axial, reciprocating motion, the headpiece distally projects from a distal end of the distal section.

According to some embodiments, the surgical tool further includes a stopper mechanism configured to restrict axial displacement of the pushrod in at least the distal direction.

According to some embodiments, the pushrod includes a pinhole extending from one side-surface thereof to an opposite side-surface thereof The stopper mechanism includes a pin extending through the pinhole from one sidewall of the distal section to an opposite sidewall thereof The pinhole may have a diameter greater than the pin, thereby restricting axial displacement of the pushrod while allowing for axial reciprocating motion of the pushrod.

According to some embodiments, the surgical tool further includes a linear-motion bearing, positioned at the distal end of the distal section such that the pushrod extends therethrough. The linear-motion bearing is configured to facilitate axial, reciprocating motion of the pushrod.

According to some embodiments, the motion converter includes a sleeve element mountable on or insertable into the distal section. The sleeve element includes the pushrod which is at least partially disposed within and along the sleeve element.

According to some embodiments, the pushrod includes a pinhole extending from one side-surface thereof to an opposite side-surface thereof The stopper mechanism includes a pin extending through the pinhole from one sidewall of the sleeve element to an opposite sidewall thereof The pinhole may have a diameter greater than the pin, thereby restricting axial displacement of the pushrod while allowing for axial reciprocating motion of the pushrod.

According to some embodiments, the cam includes one or more lobes, projecting from a distal end of the cam, such as to engage a proximal end of the pushrod when the cam effects rotary motion.

According to some embodiments, a distal end of the cam defines an oblique surface, the cam being thereby configured to engage a proximal end of the pushrod when the cam effects rotary motion.

According to some embodiments, the surgical tool is configured to effect the axial, reciprocating motion of the headpiece at rate of at least about 10,000 SPM when the angle is smaller than 180° and the cable is bent at a bend radius below 10 mm.

According to some embodiments, the surgical tool is configured to operate such that the cable rotates at a rate of at least about 10,000 revolutions per minute (RPM).

According to some embodiments, the surgical tool is further configured to effect axial, reciprocating motion such as to allow hammering a rate of up to about 240,000 SPM.

According to some embodiments, the cable includes a plurality of braided/intertwined/stranded wires.

According to some embodiments, the hard tissue is bone tissue.

According to some embodiments, the surgical tool further includes a handle configured to facilitate operation and control of the surgical tool by an operator.

According to some embodiments, a distal tip of the headpiece includes an eroding surface, is one or more protrusions, and configured for hammering hard tissue.

According to some embodiments, a circumferential surface of the headpiece is eroding, the surgical tool being thereby further configured for debulking hard tissue by grating.

Certain embodiments of the present disclosure may include some, all, or none of the above advantages. One or more other technical advantages may be readily apparent to those skilled in the art from the figures, descriptions, and claims included herein. Moreover, while specific advantages have been enumerated above, various embodiments may include all, some, or none of the enumerated advantages.

Unless otherwise defined, all technical and scientific terms used herein have the same meaning as commonly understood by one of ordinary skill in the art to which this disclosure pertains. In case of conflict, the patent specification, including definitions, governs. As used herein, the indefinite articles “a” and “an” mean “at least one” or “one or more” unless the context clearly dictates otherwise.

### BRIEF DESCRIPTION OF THE FIGURES

Some embodiments of the disclosure are described herein with reference to the accompanying figures. The description, together with the figures, makes apparent to a person having ordinary skill in the art how some embodiments may be practiced. The figures are for the purpose of illustrative description and no attempt is made to show structural details of an embodiment in more detail than is necessary for a fundamental understanding of the disclosure. For the sake of clarity, some objects depicted in the figures are not to scale.

In the figures:

FIG. 1A is a schematic, perspective view of a surgical tool configured for hard tissue removal, according to some embodiments;

FIG. 1B is a schematic, cross-sectional view of the surgical tool of FIG. 1A, according to some embodiments;

FIG. 1C is a schematic, perspective view of a distal portion and a headpiece of the surgical tool of FIG. 1A, according to some embodiments;

FIG. 1D is a schematic, perspective view of a motion converter and the headpiece of the surgical tool of FIG. 1A, according to some embodiments;

FIG. 1E is a schematic, perspective view of a distal portion of a surgical tool configured for hard tissue removal, according to some embodiments;

FIG. 1F is a schematic, cross-sectional view of a specific embodiment of the surgical tool of FIG. 1A;

FIG. 2 is a schematic, cutaway view of a distal portion of a surgical tool for hard tissue removal, according to some embodiments;

FIG. 3A is a schematic, perspective view of a surgical tool for hard tissue removal, according to some embodiments; and

FIG. 3B is a schematic, perspective view of a motion converter of the surgical tool of FIG. 3A, according to some embodiments.

### DETAILED DESCRIPTION

The principles, uses, and implementations of the teachings herein may be better understood with reference to the accompanying description and figures. Upon perusal of the description and figures present herein, one skilled in the art will be able to implement the teachings herein without undue effort or experimentation. In the figures, same reference numerals refer to same parts throughout.

In the description and claims of the application, the words “include” and “have”, and forms thereof, are not limited to members in a list with which the words may be associated.

In the description and claims of the application, the expression “at least one of A and B”, (e.g. wherein A and B are elements, method steps, claim limitations, etc.) is equivalent to “only A, only B, or both A and B”. In particular, the expressions “at least one of A and B”, “at least one of A or B”, “one or more of A and B”, and “one or more of A or B” are interchangeable.

As used herein, the term “about” may be used to specify a value of a quantity or parameter (e.g. the length of an element) to within a continuous range of values in the neighborhood of (and including) a given (stated) value. According to some embodiments, “about” may specify the value of a parameter to be between 80% and 120% of the given value. For example, the statement “the length of the element is equal to about 1 m” is equivalent to the statement “the length of the element is between 0.8 m and 1.2 m”. According to some embodiments, “about” may specify the value of a parameter to be between 90% and 110% of the given value. According to some embodiments, “about” may specify the value of a parameter to be between 95% and 105% of the given value.

As used herein, according to some embodiments, the terms “substantially” and “about” may be interchangeable.

For ease of description, in some of the figures a three-dimensional cartesian coordinate system (with orthogonal axes x, y, and z) is introduced. It is noted that the orientation of the coordinate system relative to a depicted object may vary from one figure to another.

Further, the symbol [symbol] may be used to represent an axis pointing “out of the page”, while the symbol [symbol] may be used to represent an axis pointing “into the page”.

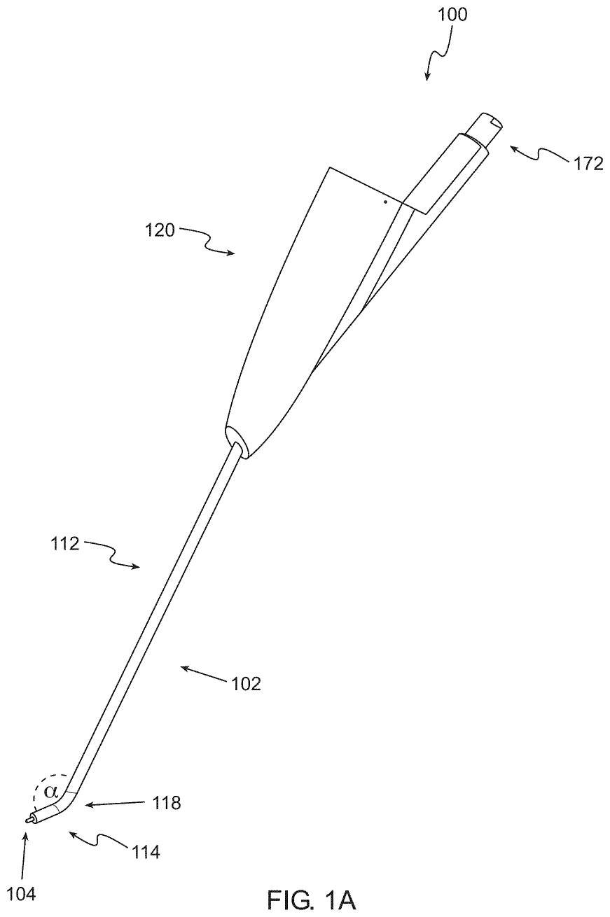

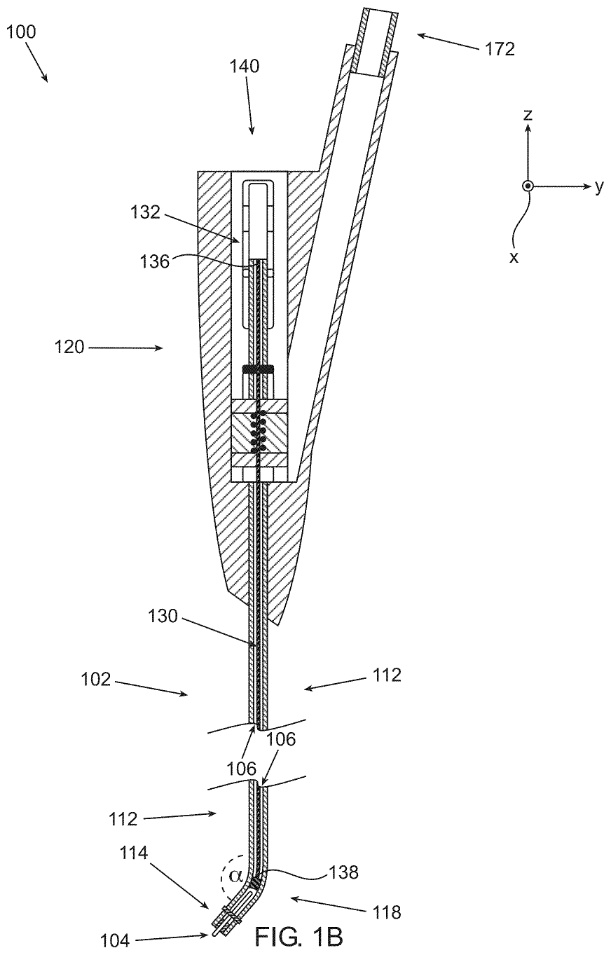

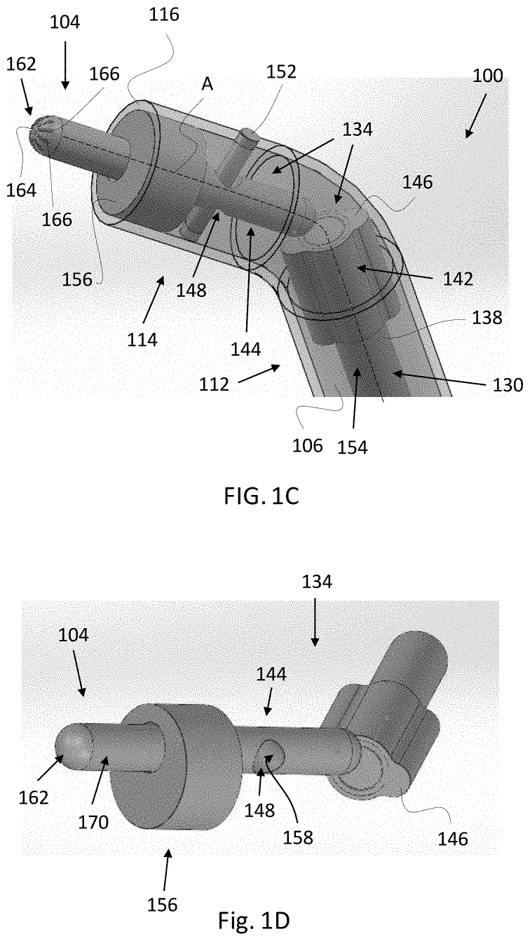

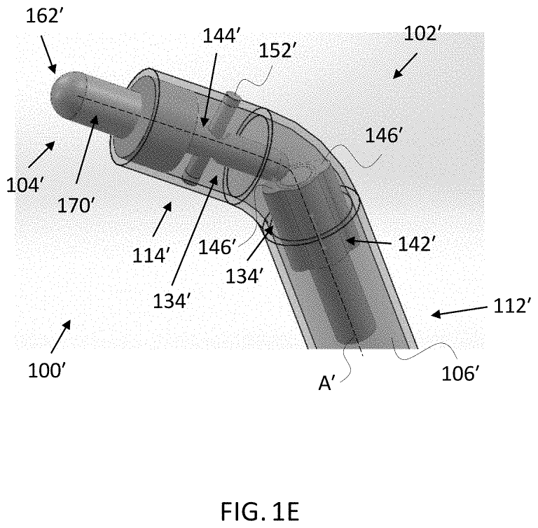

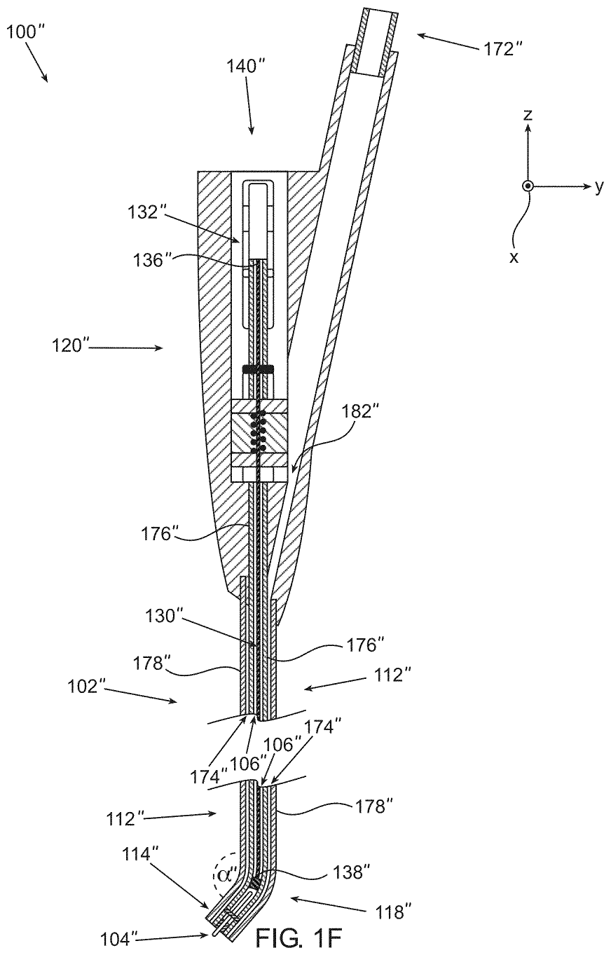

FIG. 1A schematically depicts a surgical tool 100 for hard/hardened tissue (e.g. bone) removal, according to an aspect of some embodiments. Surgical tool 100 includes a hollow member 102, which is elongated, and a headpiece 104. According to some embodiments, hollow member 102 may be tubular (i.e. the lateral cross-section thereof may be circular, cylindrical, square, rectangular, or polygonal) and may include at least one lumen 106 (shown in FIG. 1B) extending distally along a length of hollow member 102 from a proximal end of hollow member 102. Hollow member 102 includes a main section 112 and a distal section 114 positioned distally relative to main section 112 and at an angle α relative thereto (that is, main section 112 and distal section 114 define the angle α there between). According to some embodiments, distal section 114 is joined to main section 112 or detachably connected thereto. Surgical tool 100 may be configured to allow (distal) projection of headpiece 104 from a distal end 116 (indicated in FIG. 1C) of distal section 114, and, in particular, a change in the extent of the distal projection (e.g.

when headpiece 104 effects axial, reciprocating motion, as described below). According to some embodiments, headpiece 104 is exposed on or (distally) projects from distal end 116. According to some embodiments, main section 112 and distal section 114 are joined, or detachably coupled, by a bent section 118 (e.g. a curved portion or a portion which is partially folded).

According to some embodiments, main section 112 and bent section 118 are not distinct in the sense that a distal end portion of main section 112 and a proximal end portion of bent section 118 are one and the same. Additionally, or alternatively, according to some embodiments, bent section 118 and distal section 114 are not distinct in the sense that a distal end portion of bent section 118 and a proximal end portion of distal section 114 are one and the same.

According to some embodiments, wherein (i) the distal portion of main section 112 and the proximal portion of bent section 118 are one and the same, and (ii) the distal portion of bent section 118 and the proximal portion of distal section 114 are one and the same, bent section 118 may be sharply bent.

According to some embodiments, and as depicted in FIG. 1A, surgical tool 100 further includes a handle 120 configured to facilitate operation and control of surgical tool 100 (e.g. by a surgeon), as elaborated on below. According to some embodiments, and as depicted in FIG. 1B, main section 112 may extend into handle 120.

According to some embodiments, the diameter of hollow member 102 is between about 2 mm and about 10 mm. According to some embodiments, wherein the diameter of hollow member 102 varies along the length thereof, the minimum diameter is greater than about 2 mm and the maximum diameter is smaller than about 10 mm. According to some embodiments, the diameter of hollow member 102, or the maximum diameter thereof, is about 3 mm, about 4 mm, about 5 mm, about 6 mm, about 7 mm, about 8 mm, about 9 mm, or about 10 mm. Each possibility corresponds to separate embodiments. According to some embodiments, the length of hollow member 102 is between about 100 mm and about 500 mm. According to some embodiments, the length of hollow member 102 is between about 150 mm and about 250 mm. According to some embodiments, the length of hollow member 102 is about 100 mm, about 150 mm, about 200 mm, about 250 mm, about 300 mm, or about 420 mm. Each possibility corresponds to separate embodiments.

According to some embodiments, the angle a between main section 112 and distal section 114 may be between about 90° and about 120°, about 130°, about 135° about 140°, about 150°, about 160°, about 165°, about 170°, or about 180°. Each possibility corresponds to separate embodiments. According to some embodiments, hollow member 102, and, in particular, bent section 118, are characterized by a bend radius of about 25 mm, about 18 mm, about 15 mm, about 12 mm, about 9 mm, about 7 mm, or even about 5 mm. Each possibility corresponds to separate embodiments. According to some embodiments, the bend radius may be between about 2 mm and about 4 mm.

According to some embodiments, the dimensions and shape of surgical tool 100—particularly, the dimensions and shapes of hollow member 102 and headpiece 104—are such as to facilitate a “low profile”, safe insertion of headpiece 104/distal section 114 in between two adjacent vertebrae and allow access to osteophytes located underneath a vertebra. Specifically, the length and diameter of distal section 114, and the angle a between main section 112 and distal section 114 (e.g. the amount of curving of bent section 118) are such as to facilitate a low profile, safe insertion of headpiece 104/distal section 114. Further, according to some embodiments, and as depicted in FIG. 1D, headpiece 104 is excentric, in the sense of being offset relative to a central (also referred to as “longitudinal”) axis A (indicated in FIG. 1C) of lumen 106. According to some embodiments, the offsetting may facilitate safe insertion of headpiece 104.

Referring also to FIG. 1B, FIG. 1B is a cross-sectional view of surgical tool 100, according to some embodiments. The cross-section lies on a plane parallel to the yz-plane. According to some embodiments, surgical tool 100 further includes a cable 130, which is elongated, a rotation actuator 132, and a motion converter 134. Cable 130 may be disposed within (or at least partially disposed within), and along, hollow member 102, e.g. along lumen 106. According to some embodiments, rotation actuator 132 is housed within, or at least partially housed within, handle 120. Motion converter 134 may be housed within distal section 114 and bent section 118, and, optionally, hollow member 102 (i.e. such that one or more parts thereof are housed in distal section 114 and one or more other parts thereof are housed in bent section 118, and, optionally, main section 112). Cable 130 extends from a cable proximal end 136 to a cable distal end 138. Cable proximal end 136 is mechanically coupled to, or connected to, rotation actuator 132. Cable distal end 138 is mechanically coupled to, or connected to, motion converter 134, which is further mechanically coupled to, or connected to, headpiece 104.

Rotation actuator 132 is configured to induce rotations (i.e. revolutions) of cable 130 about a longitudinal axis of cable 130. According to some embodiments, wherein cable 130 is centralized within lumen 106, the longitudinal axis of cable 130 and the central axis A coincide. The central axis A may lie on the plane of the cross-section of FIG. 1B, in parallel to the yz-plane. It will be understood that the central axis A may be curved/bent, i.e. when lumen 106 is curved/bent. According to some embodiments, a central axis of hollow member 102 coincides with the central axis A.

Motion converter 134 (indicated in FIG. 1C) is configured to translate cable 130 rotation to headpiece 104 reciprocating motion. According to some embodiments, and as depicted in FIGS. 1A-1D, headpiece 104 reciprocating motion is axial, so that the extent of headpiece 104 projection from distal end 116 may alternately (i.e. by turns) increase and decrease.

According to some embodiments, rotation actuator 132 may be an electrical motor. According to some such embodiments, handle 120 may include an electrical port 140, which is electrically coupled to rotation actuator 132, thereby allowing to power rotation actuator 132 using an electrical power source external to surgical tool 100, and, in particular, handle 120. More generally, rotation actuator 132 may include any combination of mechanical components configured to tightly grip cable 130 (e.g. at, or near, cable proximal end 136), such as to allow high-frequency rotation of cable 130 about the longitudinal axis thereof.

Referring also to FIGS. 1C and 1D, FIG. 1C depicts motion converter 134 disposed within hollow member 102, and FIG. 1D is a perspective view of motion converter 134 and headpiece 104, according to some embodiments. More specifically, in FIG. 1C, the walls of hollow member 102 are depicted as semi-transparent to facilitate the description (so that components within hollow member 102, such as motion convertor 134, are visible). Motion converter 134 includes a cam 142 and a pushrod 144 (i.e. a cam follower/tracker) associated with cam 142. According to some embodiments, and as depicted in the figures, cam 142 may be mounted on/joined to cable distal end 138. Cam 142 includes one or more lobes 146 (for example, two lobes, as depicted in FIGS. 1C and 1D), or a tilted plane, configured to engage the proximal end (not numbered) of pushrod 144. Pushrod 144 is disposed in parallel, or substantially in parallel, to the central axis A and may be displaced (shifted) relative thereto, such as to allow/facilitate engagement of the proximal end of pushrod 144 by lobes 146, when cam 142 effects rotatory motion about the central axis A.

According to some embodiments, pushrod 144 includes headpiece 104. That is, a distal portion of pushrod 144 is constituted by headpiece 104. According to some alternative embodiments (not depicted in FIGS. 1A-1D), pushrod 144 is mechanically associated with headpiece 104 such that axial, reciprocating motion of pushrod 144 induces an axial, reciprocating motion of headpiece 104.

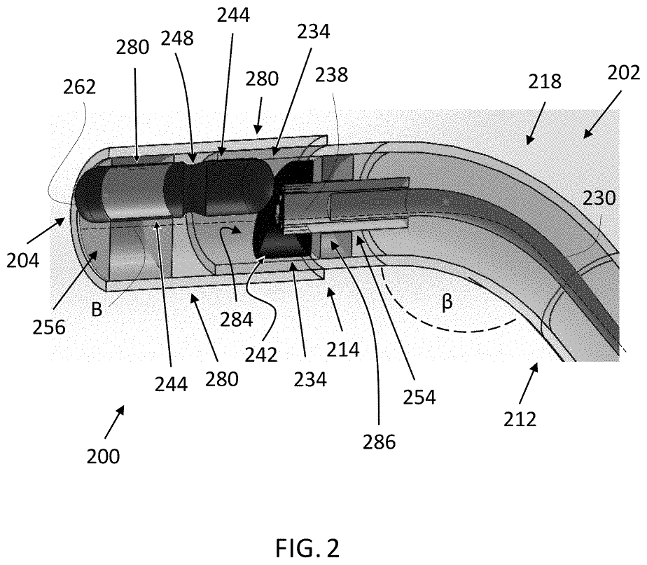

According to some embodiments, not depicted in FIGS. 1A-1D, instead of including lobes 146, the distal end (not numbered) of cam 142 may be oblique (essentially as depicted in FIG. 2). That is, in such embodiments the distal end of cam 142 defines a slanting surface (in the sense of not being perpendicular to the central axis A), which engages the proximal end of pushrod 144 (which may be displaced relative to the central axis A as described above) when cam 142 effects rotary motion about the central axis A.

Axial displacement/motion of pushrod 144 may be restricted by a stopper mechanism. More specifically, according to some embodiments, pushrod 144 includes a pinhole 148 extending from one side-surface (not numbered) of pushrod 144 to an opposite side-surface (not numbered) thereof, e.g. perpendicularly, or substantially perpendicularly, to the central axis A. A pin 152 extends through pinhole 148 and is connected on the ends thereof to opposite sidewalls (not numbered) of distal section 114. Pinhole 148 is of a greater diameter than pin 152. The greater diameter of pinhole 148 allows for (limited) axial motion of pushrod 144. However, the motion of pushrod 144, particularly in the axial direction, is restricted by pin 152, which effectively functions as a stopper preventing axial displacement of pushrod 144, and consequently of headpiece 104, beyond a preset/fixed extent.

According to some embodiments, a distal portion (including cable distal end 138) of cable 130 is rigid, such as to facilitate the mounting of cam 142 on cable distal end 138. According to some such embodiments, cable 130 includes a tube element 154, into which a flexible portion of cable 130 extends and is attached thereto (such that rotations of the flexible portion induce same and simultaneous rotations of the tube). Cam 142 may be configured to be mounted on tube element 154.

According to some embodiments, distal section 114 includes a (linear-motion) bearing 156 positioned at distal end 116. Pushrod 144, and, more precisely, a distal portion thereof (not numbered), may be disposed through bearing 156, which is configured to facilitate pushrod 144 axial reciprocating motion. (According to some embodiments, headpiece 104 may be positioned distally relative to bearing 156, at least during part of the time when headpiece 104 effects reciprocating motion.)

In operation, when cam 142 effects rotary motion, each of lobes 146, upon striking pushrod 144, distally pushes pushrod 144 until a pinhole proximal wall 158 (i.e. the proximal wall of pinhole 148) strikes (hits) pin 152. The impact of the strike reverses the direction of motion of pushrod 144, propelling (sending) pushrod 144 in the proximal direction (at least when the RPM is 4,000 or greater). Pushrod 144 may then be struck again by a lobe (of lobes 146), and the motion is then repeated, so that pushrod 144, and consequently headpiece 104, effect axial, reciprocating motion.

According to some alternative embodiments, pin 152 may be elastic so that when pressed by pushrod 144—due to the elasticity of pin 152—pin 152 pushes back pushrod 144, reversing the direction of motion thereof (and of headpiece 104). In such embodiments, pinhole 148 need not have a larger, or substantially larger, diameter than the diameter of pin 152. Alternatively, pushrod 144 may include or constitute a resilient member (e.g. a spring) mechanically associated with headpiece 104. The resilient member may be configured to induce and control headpiece 104 reciprocating motion.

According to some embodiments, headpiece 104 is configured for breaking up/fragmenting hard/rigid (i.e. non-elastic) tissue by striking/hitting/hammering/grinding the tissue. In particular, a distal tip 162 of headpiece 104 may be configured for hammering hard tissue. Distal tip 162 may be rounded, as depicted in FIG. 1C, or flat or substantially flat (e.g. including a flat or even a concave surface perpendicular to a longitudinal axis of distal section 114). According to some non-limiting embodiments, and as depicted in FIG. 1C, distal tip 162 may include an eroding surface 164 configured for breaking up hard tissue. Eroding surface 164 may include protrusions 166 (shown in FIG. 1C; not all of which are numbered) configured to facilitate the breaking up of hard tissue. Protrusions 166 may be formed, for example, by diamonds embedded in eroding surface 164.

According to some embodiments, eroding surface 164 may be curved, e.g. in the form of a cap. A circumferential surface 170 of headpiece 104 (indicated in FIG. 1D) is positioned proximally to eroding surface 164. According to some embodiments, circumferential surface 170 may be configured for reciprocating motion in-and-out of bearing 156 during surgical tool 100 operation.

According to some embodiments, lumen 106 may follow the curvature of hollow member 102 and may have a diameter between about 30% and about 100% greater than the diameter of cable 130, thereby ensuring that cable 130 (particularly, the flexible portion(s) thereof, described below) does not kink or warp within lumen 106. According to some such embodiments, the diameter of lumen 106 is, for example, between about 1 mm and about 4 mm (e.g. 2.8 mm). Lumen 106 may optionally be centered within hollow member 102 (i.e. such that the longitudinal axes thereof overlap).

Cable 130 may include a rigid portion (e.g. tube element 154) and a flexible portion (not numbered). The rigid portion may be connected to the flexible portion (e.g. via crimping or welding). The rigid portion may extend along a straight segment of lumen 106 (e.g. in main section 112), while the flexible portion may extend along bent section 118, and, optionally, along one or more straight segments of lumen 106. As elaborated on below, cable 130 may include a rigid tube crimped over an end thereof.

According to some embodiments, cable 130 may be configured for high torsional rigidity and low bending rigidity, such as to allow cable 130 to be rotated about the longitudinal axis thereof at high rotation frequencies while bent at a small radius of curvature. In particular, surgical tool 100 is configured to enable the transfer of torque (from rotation actuator 132 to cam 142) when cable 130 is bent. Advantageously, this allows for effecting high frequency axial, reciprocating motion of headpiece 104 against hard tissue located at difficult-to-reach anatomical sites (e.g. between vertebrae), which require surgical tool 100 to be bent (in order to reach the anatomical site). High torsional rigidity and low bending rigidity facilitate rotary motion of a wire/cable when bent/curved. Having a low bending rigidity (bending stiffness) potentially allows for low bending-related stress and higher resistance to fatigue which may result from high rotation frequencies and/or high torques.

According to some embodiments, cable 130 includes a core including a plurality of intertwined wires configured for maintaining structural integrity and low bending stress. Cable 130 further includes one or more outer layers, configured for transferring torque. According to some embodiments, the plurality of intertwined wires of the core may be braided, stranded and/or twisted. (In particular, according to some embodiments, the core does not consist of a single solid core (e.g. an elongated shaft, a mandrel). The outer layers may be coiled, winded, or twisted, with adjacent layers being coiled/winded in opposite senses—cable 130 being thereby adapted to transfer torque in both senses (e.g. “right-handed” torque and “left-handed” torque). According to some embodiments, at least some adjacent layers (of the outer layers) may be winded in the same sense.

According to some exemplary embodiments, the core of cable 130 may be fabricated, for example, from seven 304V stainless-steel wires (each having a diameter of, for example, between about 0.07 mm and about 0.1 mm, e.g. 0.084 mm) twisted and/or intertwined into a rope. More generally, the core of cable 130 may be fabricated, for example, from wires of any of the 300 stainless series and/or Nitinol. Several layers of coils, e.g. three layers, may be wound around the core. Each successive coil may optionally be wound in an opposite direction to (the winding of) the coil directly beneath it. The inner coil (closest to cable's inner core) may include, for example, five wires (with a diameter of e.g. about 0.12 mm each), the middle coil may include, for example, five wires (with a diameter of e.g. about 0.14 mm each), and the outer coil may include, for example, five wires (with a diameter of e.g. about 0.16 mm each).

Cable 130 design is configured to enable the transfer of rotational and longitudinal motion, i.e. torque and rotational frequency and axial force and speed, along curved paths, including highly curved paths, in a manner resistant to fatigue. Such paths may be fixed, as in embodiments wherein bent section 118 is rigid, or variable, as in embodiments wherein bent section 118 is flexible and may conform to a range of angles or curvatures before and/or during the surgery.

According to some embodiments, cable 130 has a diameter between about 0.3 mm and about 5 mm, e.g. about 0.5 mm, about 1.5 mm, or about 3 mm.

According to some embodiments, cable 130 may be configured to allow rotation frequencies of up to about 120,000 RPM (about the longitudinal axis thereof) and to be acted on by, and to apply, torques of at least about 5 N·cm (newton-centimeter). According to some embodiments, cable 130 may be configured to allow rotation frequencies of up to about 120,000 RPM for both clockwise and anti-clockwise rotations about the longitudinal axis thereof. According to some embodiments, cable 130 may be configured to be acted on by, and to apply, torques of about 5 N·cm or about 7 N·cm.

According to some embodiments, to facilitate operation of surgical tool 100, handle 120 may be shaped substantially as an inverted cone. According to some embodiments, the handle may have a length, for example, between about 50 mm and about 105 mm, a proximal diameter, for example, between about 10 mm and about 30 mm, and a distal diameter, for example, between about 5 mm and about 30 mm. Handle 120 may be fabricated as a shell composed of one or more cast, machined, or injection-molded pieces.

According to some embodiments, handle 120 includes an operational input 172 (also referred to as a suction, irrigation and/or working channel). According to some embodiments, operational input 172 may be fluidly coupled to lumen 106 via a channel (not shown), thereby allowing, for example, to irrigate a target tissue site in order to cool or wash the target tissue site. According to some embodiments, operational input 172 may function as a port for introducing/withdrawing/suction of fluids. In particular, operational input 172 may be configured to be coupled to a vacuum pump (e.g. by having inserted thereinto a suction tip).

According to some embodiments, a wall of hollow member 102 may have embedded therein a second lumen (not shown) extending along main section 112 from a distal tip of hollow member 102 onto handle 120. The second lumen may be fluidly coupled to operational input 172 via a channel (not shown) in handle 120. In such embodiments, operational input 172 may additionally or alternatively be used to introduce additional surgical instruments (e.g. a tissue dissector, a spatula, an electrophysiological monitoring device/neuro-stimulation device, sensors/cameras, and/or the like).

According to some embodiments, a distal tip of hollow member 102 may include or form an electrode. For example, distal end 116 may include or form an electrode. According to some embodiments, an electrical wire (not shown) may be embedded within the wall of hollow member 102, such that a distal end of the wire is connected to the electrode and a proximal end of the wire is configured to be coupled to an electrical power source (e.g. via electrical port 140 or operational input 172). The electrode may be used in conjunction with a second electrode placed on the body of a subject (during a surgical procedure wherein surgical tool 100 is inserted into the body of the subject) to provide neuro-stimulation at or near a target tissue site. According to some embodiments, wherein at a least part of hollow member 102 is made of an electrically conducting material (e.g. stainless steel), hollow member 102 may be used to establish a voltage difference between the electrode and the second electrode (so that the necessity of using an electrical wire at least along hollow member 102 is obviated).

According to some embodiments, a distal tip of hollow member 102 may include or form two electrodes. For example, distal end 116 may include or form two electrodes. According to some embodiments, electrical wires (not shown) may be embedded within the wall of hollow member 102, such that distal ends of the wires are connected to the two electrodes, respectively, and proximal ends of the wires are configured to be coupled to an electrical power source (e.g. via electrical port 140 or operational input 172). The electrodes may be used to establish a voltage difference there between, such as to allow for localized neuro-stimulation within the body of a subject (during a surgical procedure) at or near a treatment site. According to some embodiments, wherein at least a part(s) of hollow member 102 is made of an electrically conducting material (e.g. stainless steel), hollow member 102 may be used to establish the voltage difference between the two electrodes.

According to some embodiments, handle 120 may include a user interface for operating rotation actuator 132, setting rotation actuator 132 motor parameters (e.g. RPM and sense of rotation), setting the debulking time, operating and setting irrigation parameters, as well as controlling adjunct devices such as a neuro-stimulation device. The user interface may also include a display for presenting various parameters related to rotation actuator 132 operation or to irrigation, as well as information indicative of headpiece 104 and bent section 118 status and condition, such as temperature thereof, mechanical integrity thereof, headpiece 104 position, and the like. According to some embodiments, the user interface may also be configured to display information related to one or more adjunct devices (e.g. an electrophysiological monitoring device) used during a debulking procedure.

Furthermore, handle 120 may be designed and configured such that a surgeon is able to maintain a clear line-of-site along surgical tool 100 (and, in particular, hollow member 102), so as to allow the surgeon to monitor progress while debulking target tissue and to avoid damaging surrounding tissue (which is not intended for removal).

FIG. 1E schematically depicts a distal portion of a surgical tool 100′, according to some embodiments. Surgical tool 100′ includes a hollow member 102′, which may be similar to hollow member 102, and a headpiece 104′ configured for breaking up hard tissue by the grating (e.g. filing, polishing) thereof (in addition to, or alternatively to, breaking up the tissue by the hammering/grounding thereof), as explained below. According to some embodiments, wherein headpiece 104′ may be utilized both for grating tissue and hammering tissue, surgical tool 100′ may be a specific embodiment of surgical tool 100.

To facilitate the description, in FIG. 1E the walls of hollow member 102′ are depicted as semi-transparent, so that components within hollow member 102, such as a motion convertor 134′ (which may be similar to motion converter 134), are visible. According to some embodiments, and as depicted in FIG. 1E, a circumferential surface 170′ (e.g. a cylindrical surface) of headpiece 104′ is eroding (e.g. rough-textured at least on a distal portion thereof), being thereby configured for grating/fragmenting hard tissue. More specifically, in these embodiments, by bringing surgical tool 100′ against a hard tissue surface such that circumferential surface 170′ is adjacent to the tissue surface and is in contact therewith, and activating surgical tool 100′, due to headpiece 104′ axial, reciprocating motion, circumferential surface 170′ grates the tissue surface (by moving back and forth in the axial direction), leading to fragmentation of the tissue.

Also indicated are a cam 142′ and a pushrod 144′, which is configured to function as a cam follower/tracker. According to some embodiments, and as depicted in FIG. 1E, cam 142′ may include lobes 146′. According to some alternative embodiments, not depicted in FIG. 1E, a distal surface of cam 142′ may be oblique. According to some embodiments, and as depicted in FIG. 1E, pushrod 144′ includes headpiece 104′. Alternatively, pushrod 144′ may be mechanically associated with headpiece 104′ such that axial, reciprocating motion of pushrod 144′ induces an axial, reciprocating motion of headpiece 104′. Surgical tool 100′ is configured such that rotary motion of cam 142′ is translated into axial, reciprocating motion of pushrod 144′ (and headpiece 104′), essentially as described above with respect to surgical tool 100.

According to some embodiments, surgical tool 100′ is a specific embodiment of surgical tool 100, and is configured for breaking up tissue both by hammering and by grating. In such embodiments, both a distal tip 162′ of headpiece 104′ and circumferential surface 170′ are eroding (e.g. rough-textured, with distal tip 162′ optionally including protrusions (not shown in FIG. 1E) such as protrusions 166).Further indicated in FIG. 1E are a lumen 106′, a distal section 114′, and a pin 152′ (which may be respectively similar to lumen 106, distal section 114, and pin 152 of a surgical tool 100), and a central axis A′ of lumen 106′.

FIG. 1F is a cross-sectional view of a surgical tool 100″, according to some embodiments. Surgical tool 100″ constitutes a specific embodiment of surgical tool 100. Surgical tool 100″ includes a pair of lumens, as described below. More specifically, surgical tool 100″ includes a hollow member 102″, a headpiece 104″, and a handle 120″, which are specific embodiments of hollow member 102, headpiece 104, and handle 120, respectively. Hollow member 102″ includes an inner lumen 106″ (which is a specific embodiment of lumen 106) and an outer lumen 174″ disposed about inner lumen 106″, as described below. Inner lumen 106″ is defined by an inner wall 176″ (e.g. a cylindrical wall), and outer lumen 174″ is defined by an outer wall 178″ (e.g. a cylindrical wall having a greater radius than inner wall 176″) disposed about inner wall 176″. Inner lumen 106″ includes a cable 130″ disposed therein and extending there along. Cable 130″ extends from a cable proximal end 136″ to a cable distal end 138″ and constitutes a specific embodiment of cable 130. Outer lumen 174″ may be fluidly-coupled to an operational input 172″ (which is a specific embodiment of operational input 172) via a channel 182″ in handle 120″, thereby allowing introduction and/or withdrawal/suction of fluids via operational input 172″, as well as introduction of additional surgical instruments, essentially as described above in the description of surgical tool 100.

Also indicated are a main section 112″, a distal section 114″, and a bent section 118″ of hollow member 102″, and a rotation actuator 132″, which are specific embodiments of main section 112, distal section 114, bent section 118, and rotation actuator 132, respectively.

According to some embodiments, a distal tip (not indicated) of outer wall 178″ may form a first electrode while a distal tip (not indicated) of inner wall 176″ may form a second electrode, such as to allow for in vivo neuro-stimulation (and neuro-monitoring) during a surgical procedure wherein bone tissue is removed from a site including one or more nerves. According to some embodiments, each of the walls may include, embedded therein, an electrical wire (not shown) connected on a distal end thereof to the respective electrode and electrically coupled on a proximal end thereof to an electrical port 140″ (which is a specific embodiment of electrical port 140) or operational input 172″, such as to allow establishing a voltage between the electrodes. According to some embodiments, each of outer wall 178″ and inner wall 176″ may be made of an electrically conducting material, thereby obviating usage of electrical wires embedded within the walls.

FIG. 2 schematically depicts a surgical tool 200 (not fully shown) for hard tissue removal, according to some embodiments. Surgical tool 200 includes a hollow member 202 (not fully shown) and a headpiece 204 similar to hollow member 102 and headpiece 104, respectively. Hollow member 202 includes a main section 212 (not fully shown) and a distal section 214, which is distally positioned relative to main section 212 at an angle β relative thereto, and joined thereto (e.g. via a bent section 218 of hollow member 202). According to some embodiments, β may be between about 90° and about 120°, about 130°, about 135°, about 140°, about 150°, about 160°, about 165°, about 170°, or about 180°. Each possibility corresponds to separate embodiments.

According to some embodiments, main section 212 and bent section 218 are not distinct in the sense that a distal end portion of main section 212 and a proximal end portion of bent section 218 are one and the same. Additionally, or alternatively, according to some embodiments, bent section 218 and distal section 214 are not distinct in the sense that a distal end portion of bent section 218 and a proximal end portion of distal section 214 are one and the same.

According to some embodiments, wherein (i) the distal portion of main section 212 and the proximal portion of bent section 218 are one and the same, and (ii) the distal portion of bent section 218 and the proximal portion of distal section 214 are one and the same, bent section 218 may be sharply bent.

Surgical tool 200 further includes a cable 230 similar to cable 130, a rotation actuator (not shown) similar to rotation actuator 132, and a motion converter 234. Cable 230 and the rotation actuator may be mechanically coupled essentially as described above with respect to cable 130 and rotation actuator 132. Motion converter 234 may be installable in and/or on distal section 214. More specifically, motion converter 234 includes a cam 242, a pushrod 244 (i.e. a cam follower/tracker), and a sleeve element 280, which may be tubular, having a circular or oval transverse cross-section, or have or rectangular or polygonal transverse cross-section. Sleeve element 280 includes pushrod 244, which is disposed along the length of sleeve element 280 (in parallel to a central axis B of distal section 214, which may also be shared by sleeve element 280). Cam 242 may be positioned within distal section 214 and may be installable/mountable on a cable distal end 238 (i.e. the distal end of cable 230), such that rotation of cable 230 induces (an equal or substantially equal) rotation of cam 242.

According to some embodiments, sleeve element 280 may be installable/mountable on/in distal section 214, such as to be concentrically disposed thereon/therein, and such as to allow pushrod 244 to be engaged by cam 242, as elaborated on below. According to some such embodiments, sleeve element 280 may be slid on distal section 214 (and thereby coupled thereto). According to some embodiments, and as depicted in FIG. 2, sleeve element 280 is of a slightly greater diameter than distal section 214, and as such is configured to be mounted on distal section 214. According to some such embodiments, sleeve element 280 is of a slightly smaller diameter than distal section 214, and as such is configured to be partially inserted into distal section 214.

According to some alternative embodiments, sleeve element 280 may be non-removably affixed to on distal section 214 or may be integrally formed therewith.

Similarly to pushrod 144, pushrod 244 may include a pinhole 248 extending from one side-surface of pushrod 244 to an opposite side-surface thereof, e.g. perpendicularly, or substantially perpendicularly, to the central axis B. A pin (not shown) extends through pinhole 248 and is connected on the ends thereof to opposite sidewalls (not numbered) of sleeve element 280. Pinhole 248 may be of a greater diameter than the pin, such as to allow for (limited) axial motion of pushrod 244, essentially as described above with respect to motion converter 134 and as explained below.

According to some embodiments, and as depicted in FIG. 2, a cam distal end 284 (i.e. the distal end of cam 242) may be oblique (i.e. defining a slanting surface) in the sense of not being perpendicular to longitudinal axis B. Pushrod 244 may be displaced relative to the central axis B (e.g. in parallel, or substantially in parallel, thereto), such that by starting at a configuration, wherein pushrod 244 is proximally positioned to the maximum (so that a distal wall of pinhole 248 may contact a distal wall of the pin), cam distal end 284 strikes pushrod 244 as cam 242 is rotated (about the central axis B). When so struck, pushrod 244 is sent in the distal direction until a proximal wall (not numbered) of pinhole 248 hits the pin, which sends pushrod 244 back in the proximal direction to be struck again by cam 242, and so on and so forth, such that axial, reciprocating motion of pushrod 244 and, hence, headpiece 204, is generated.

According to some embodiments, cable 230 includes a tube element 254 (which includes cable distal end 238) into which a flexible portion of cable 230 extends and is attached thereto (such that rotations of the flexible portion induce same and simultaneous rotations of the tube). Cam 242 may be configured to be mounted on tube element 254.

According to some embodiments, not depicted in FIG. 2, cam distal end 284 may include one or more lobes (similar to lobes 146) configured to engage pushrod 244. According to some such embodiments, cam distal end 284 is not oblique.

According to some embodiments, a distal tip 262 of headpiece 204 is configured for axial hammering of hard tissue, essentially as described above in the description of headpiece 104 of surgical tool 100. More specifically, according to some such embodiments, distal tip 262 may include an eroding surface, which may include protrusions similar to protrusions 166 of distal tip 162.

Additionally, or alternatively, according to some embodiments, a circumferential surface (not numbered) of headpiece 204 may be eroding (e.g. rough-textured at least on distal portions thereof), essentially as described above with respect to circumferential surface 170′ of headpiece 104′ of surgical tool 100′. In such embodiments, in addition to, or alternatively to, breaking up hard tissue by (axial) hammering thereof, headpiece 204 (and, hence, surgical tool 200) is configured for fragmenting hard tissue by grating (when effecting axial, reciprocating motion adjacently to a surface of the hard tissue, as described above in the description of surgical tool 100).

According to some embodiments, headpiece 204 is detachably mounted on pushrod 244, such as to allow replacing headpiece 204, e.g. due to wear and tear, or in order to mount a different headpiece, of e.g. a different size, shape, and/or roughness of the eroding surfaces.

According to some alternative embodiments, headpiece 204 may form a part of pushrod 244 (for example, headpiece 204 and pushrod 244 may be integrally formed). According to some such embodiments, wherein sleeve element 280 is removable, headpiece 204 may be replaced by replacing sleeve element 280.

According to some embodiments, sleeve element 280 includes a first bearing 256 (e.g. a linear-motion bearing) and a second bearing 286 (e.g. a roller bearing). First bearing 256 may be positioned at a distal end (not numbered) of sleeve element 280. Pushrod 244, and, more precisely, a distal portion thereof (not numbered), may be disposed through first bearing 256, which is configured facilitate pushrod 244 axial, reciprocating motion. According to some embodiments, headpiece 204 may be positioned distally relative to first bearing 256, at least during part of the time when headpiece 204 effects reciprocating motion. Second bearing 286 may be positioned at a proximal end (not numbered) of distal section 214. Tube element 254 may be disposed through second bearing 286, which is configured to facilitate tube element 254 rotary motion (and, hence, the imparting of rotation from cable 230 to cam 242).

According to some embodiments, motion converter 234 is detachably mountable. That is, each of sleeve element 280 and cam 242 may be removed and replaced, thereby allowing to mount on distal section 214 a debulking element with a different function, such as a drill bit or a cutting bit, so as to allow using surgical tool 200 (omitting headpiece 204, motion converter 234, and sleeve element 280, but with a debulking element installed on distal section 214) also for e.g. drilling and/or cutting, and, in particular, for debulking soft (elastic) tissue. According to some such embodiments, surgical tool 200 may be provided as part of a kit including (in addition to headpiece 204, motion converter 234, and sleeve element 280) one or more debulking elements as described above.

According to some embodiments, and as depicted in FIG. 2, headpiece 204 is excentric, thereby potentially facilitating safe insertion of headpiece 204.

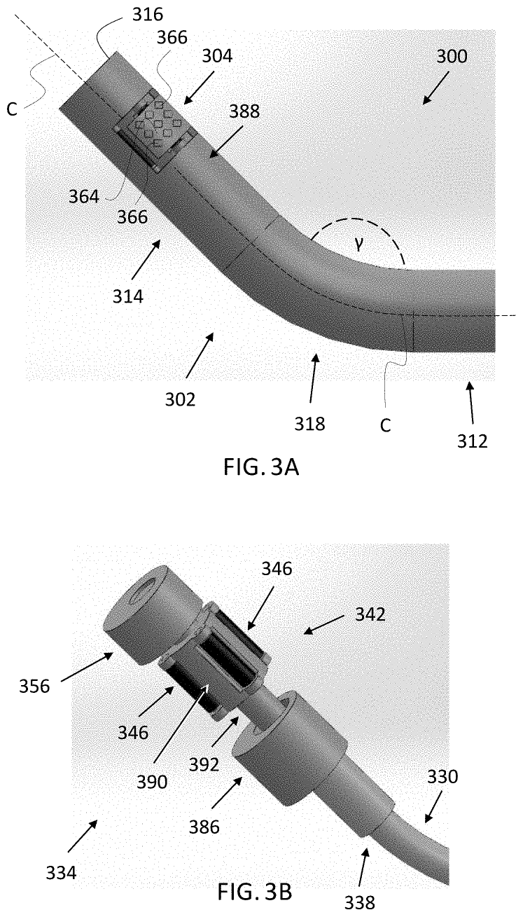

FIG. 3A schematically depicts a surgical tool 300 (of which only a distal portion is shown) for hard tissue removal, according to an aspect of some embodiments. Surgical tool 300 is similar to surgical tools 100 and 200 but differs therefrom at least in being configured for transverse (perpendicular to the axial direction) hammering/pounding of hard tissue, instead of axial hammering/pounding of hard tissue, as explained below. Surgical tool 300 includes a hollow member 302 (not fully shown), which may be similar to hollow member 102, and a work element 304 (e.g. a plate or plate-like element configured for hammering hard tissue). Hollow member 302 includes a main section 312 (not fully shown) and a distal section 314, which is distally positioned relative to main section 312, set at an angle y relative thereto, and joined thereto. According to some embodiments, y may be between about 90° and about 120°, about 130°, about 135°, about 140°, about 150°, about 160°, about 165°, about 170°, or about 180°. Each possibility corresponds to separate embodiments.

Work element 304 may form or define an eroding surface 364 configured for breaking up hard tissue by striking/repeatedly striking the tissue. According to some embodiments, and as depicted in FIG. 3A, eroding surface 364 may include protrusions 366 configured to facilitate the breaking up of the tissue. Protrusions 366 (not all of which are numbered) may be formed, for example, by diamonds embedded in eroding surface 364. According to some embodiments, apart from protrusions 366, work element 304, or at least eroding surface 364, is flat or substantially flat. According to some other embodiments, work element 304, or at least eroding surface 364 is curved (e.g. such that, except for protrusions 366, eroding surface 364 conforms to the cylindrical shape of distal section 314 when work element 304 is tucked in). Surgical tool 300 may be configured to allow transverse projection of work element 304 from a sidewall 388 of distal section 314, and, in particular, a change in the extent of the transverse projection (e.g. when work element 304 effects transverse, reciprocating motion, as described below).

Referring also to FIG. 3B, FIG. 3B provides a perspective view of a motion converter 334 of surgical tool 300. Work element 304 is also shown and a cable 330 of surgical tool 300 is shown in part. Cable 330 is similar to cable 130. Surgical tool 300 further includes a rotation actuator (not shown), which may be similar to rotation actuator 132, and is configured to induce cable 330 rotary motion. In particular, cable 330 and the rotation actuator may be mechanically coupled essentially as described above with respect to cable 130 and rotation actuator 132. Motion converter 334 is configured to translate cable 330 rotary motion into transverse (i.e. perpendicular to a central axis C of distal section 314), reciprocating motion of work element 304. According to some embodiments, motion converter 334 may be positioned in distal section 314. According to some embodiments, some parts of motion converter 334 may be positioned in distal section 314 and other parts of motion converter 334 may be positioned in a bent section 318 joining main section 312 to distal section 314. More specifically, motion converter 334 includes a cam 342 mechanically associated with work element 304. Cam 342 may include one or more lobes 346 (four, for example, in FIG. 3) radially (e.g. perpendicularly to the central axis C) extending from a cam body 390. Cam body 390 may be mounted on a shaft 392 which is mechanically coupled to a cable distal end 338 (i.e. the distal end of cable 330). Shaft 392 may extend along, substantially along, or in parallel to, or substantially in parallel to, the central axis C of distal section 314. According to some embodiments, shaft 392 may be excentric in the sense of being displaced (offset) relative the central axis C, so that cam 342 is also displaced relative to the central axis C.

According to some embodiments, main section 312 and bent section 318 are not distinct in the sense that a distal end portion of main section 312 and a proximal end portion of bent section 318 are one and the same. Additionally, or alternatively, according to some embodiments, bent section 318 and distal section 314 are not distinct in the sense that a distal end portion of bent section 318 and a proximal end portion of distal section 314 are one and the same.

According to some embodiments, wherein (i) the distal portion of main section 312 and the proximal portion of bent section 318 are one and the same, and (ii) the distal portion of bent section 318 and the proximal portion of distal section 314 are one and the same, bent section 318 may be sharply bent.

Motion converter 334 may further include a spring (not shown), such as a leaf spring, which is coupled to work element 304 (and housed within distal section 314). The spring may be configured to exert a return force on work element 304 when work element 304 is radially displaced relative to sidewall 388 (i.e. such as to project from sidewall 388).

In debulking procedure, distal section 314 may first be positioned in parallel to a target tissue surface. The distance between distal section 314 and the tissue surface is selected to be such that when work element 304 is radially projected from distal section 314 (i.e. when transverse, reciprocating motion of work element 304 is effected), eroding surface 364 will strike the tissue surface. When the rotation actuator is switched on, so that cable 330 effects rotary motion, rotary motion of shaft 392, and hence, of cam 342, is induced. As cam 342 rotates, each of lobes 346, in turn, may engage work element 304, such as to radially push work element 304, until the force of the spring pulls back work element 304, which is then engaged again by one of lobes 346, such that transverse (radial), reciprocating motion of work element 304 is generated.

According to some embodiments, distal section 314 includes a first bearing 356 (e.g. a roller bearing), positioned at a distal end 316 of distal section 314, and a second bearing 386 (e.g. a roller bearing), which may be positioned at a proximal end (not numbered) of distal section 314. Shaft 392 may be disposed through first bearing 356 and second bearing 386, which are configured to facilitate shaft 392 rotary motion (cam body 390 is positioned between first bearing 356 and second bearing 386).

According to some embodiments of the surgical tools disclosed herein (e.g. surgical tools 100, 100′, 100″, 200, and 300), the surgical tools are adapted for debulking hard tissue while leaving intact soft (elastic) tissue (e.g. the mechanical properties of the headpiece/work element and the amplitude and frequency of the reciprocating motion are such that when the headpiece/work element effects reciprocating motion against a bone, it may grind/fragment the bone, but when the headpiece/work element effects reciprocating motion against soft tissue, it will not damage it).

Thus, according to an aspect of some embodiments, there is provided a surgical tool, such as surgical tool 100 or surgical tool 200 or similar thereto, for breaking up/fragmenting hard tissue (e.g. bone tissue). The surgical tool includes a hollow member (e.g. a flexible or malleable tube), a cable, a headpiece, a rotation actuator, and a motion converter. The hollow member is elongated and includes a main section and a distal section. The distal section may be positioned at an angle relative to the main section (e.g. to facilitate accessing difficult-to-reach anatomical sites, such as between vertebrae). The cable is elongated and extends within the main section there along from a cable proximal end (i.e. the proximal end of the cable) to a cable distal end (i.e. the distal end of the cable). The cable is configured to resist helixing. The headpiece is positioned in or on the distal section and is configured for axial, reciprocating motion wherein at least some of the headpiece is exposed outside the distal section. The rotation actuator is coupled to the cable proximal end and is configured to effect rotary motion of the cable about a longitudinal axis of the cable. The motion converter is coupled to the cable distal end and to the headpiece. The motion converter is configured to transform rotary motion of the cable into the axial, reciprocating motion of the headpiece. The headpiece may be further configured to break up/fragment hard tissue by hammering of the tissue, when effecting axial, reciprocating motion, while simultaneously minimizing damage to soft tissue if struck.

According to some embodiments, wherein the distal section is positioned at an angle relative to the main section, (i) the hollow member further includes a bent section positioned between, or defined by, the main section and the distal section, (ii) the conversion of the rotary motion of the cable into axial, reciprocating motion of the headpiece is generated in the bent section or in proximity thereto. According to some embodiments, the reciprocating motion of the headpiece is generated distally to the bent section. According to some embodiments, the reciprocating motion of the headpiece is generated proximally to the bent section.

According to some embodiments, the hollow member may be configured such as to allow controllably changing the angle between the main section and the distal section.

According to some embodiments, the bent section is flexible. According some such embodiments, the bent section may be flexible such as to shape-wise adapt to physical (geometrical) constraints within a body of a subject during or a during the surgical procedure. The shape-wise adaptability may potentially aid in reaching difficult-to-access target sites.

According to some embodiments, the headpiece may be further configured such as to strike the tissue at a rate of about 10,000-480,000, 20,000-480,000, 50,000-480,000 or any other range within 10,000-480,000 strikes per minute (SPM). Without being bound by any theory, the SPM may be set according to the type of headpiece attached, e.g. whether the headpiece is smooth, eroded or serrated, coated with abrasive material etc.

According to some embodiments, at least during the axial, reciprocating motion, the headpiece (also referred to as “cutting head”) distally projects from a distal end of the distal section.

According to some embodiments, the headpiece is excentric in the sense of being laterally offset (laterally displaced) relative to a central axis of the distal section. The offsetting of the headpiece may facilitate treatment of sites to which access would otherwise be difficult or require removal of tissue blocking access to the site.

According to some embodiments, a distal tip of hollow member includes or constitutes one or more electrodes, being thereby configured for electrophysiological monitoring and/or neurostimulation. According to some such embodiments, the one or more electrodes are configured to function as, a single electrode. The hollow member is thereby configured to allow establishing a voltage between the electrode and an external electrode placed on/in a body of a subject during a hard tissue debulking procedure. According to some alternative embodiments, the one or more electrodes are configured to function as two electrodes. The hollow member being thereby configured to allow establish a voltage difference between the two electrodes.

According to some embodiments, at least part of the hollow member—including the distal tip of the hollow member—is made of an electrically conducting material, and wherein the distal tip of the hollow member constitutes the one or more electrodes.

According to some embodiments, the motion converter includes a cam and a pushrod (i.e. an elongated cam tracker/follower). The cam is mechanically coupled to the cable distal end. The pushrod is mechanically coupled to the headpiece and is configured to engage the cam and to effect axial, reciprocating motion.

According to some embodiments, the headpiece is mounted on a distal end of the pushrod. According to some such embodiments, the headpiece is detachably mounted on the distal end of the pushrod, such as to allow switching between different headpieces varying, for example, in shape and/or dimensions.

According to some embodiments, the headpiece and the pushrod are integrally formed with the headpiece constituting the distalmost part of the pushrod.

According to some embodiments, the cam is mounted on the cable distal end. According to some such embodiments, the cam is detachably mounted on the cable distal end.

According to some embodiments, the hollow member further includes a bent section joined on a first end thereof to the main section and on a second end thereof to the distal section. The cable may extend into the bent section. The cam may be housed in in the bent section.

According to some embodiments, a distal end portion of the main section and proximal end portion of the bent section are one and the same. Additionally, or alternatively, according to some embodiments, a distal end portion of the bent section and a proximal end portion of the distal section are one and the same.

According to some embodiments, wherein (i) the distal portion of the main section and the proximal portion of the bent section are one and the same, and (ii) the distal portion of the bent section and the proximal portion of the distal section are one and the same, the bent section may be sharply bent (e.g. similarly the spatial relation between two sides of a triangle).

According to some embodiments, the motion converter is housed in the distal section or is partially housed in the distal section and partially housed in the bent section.

According to some embodiments, at least a portion of the cable, which extends along the bent section, is flexible.

According to some embodiments, the surgical tool includes a stopper mechanism configured to restrict axial displacement of the pushrod in at least the distal direction. The stopper mechanism may be positioned in the distal section and/or may constitute a part of the motion converter.

According to some embodiments, the pushrod includes a pinhole (i.e. hole) extending from one side-surface thereof to an opposite side-surface thereof The stopper mechanism includes a pin extending through the pinhole from one (inner) sidewall of the distal section to an opposite (inner) sidewall of the distal section. The pinhole is characterized by a diameter which is greater than that of the pin, thereby restricting axial displacement of the pushrod while allowing for axial reciprocating motion of the pushrod. According to some such embodiments, the pin is mounted perpendicularly, or substantially perpendicularly, to the pushrod.

According to some embodiments, the distal section includes a linear-motion bearing positioned at the distal end of the distal section with the pushrod extending therethrough.

The linear-motion bearing is configured to facilitate the axial, reciprocating motion of the pushrod.

According to some embodiments, the motion converter is removably installed, thereby allowing to mount on distal section different tissue-debulking elements, and to switch there between, for example, the motion converter may be used for debulking hard tissue and replaced with a cutting element when soft (elastic) tissue needs to be debulked.

According to some embodiments, the cam is irremovably installed on the cam member distal end, while all or some of the remaining components of the motion converter are removably installed.

According to some embodiments, the motion converter further includes a sleeve element mountable on and/or in the distal section. The sleeve element includes the pushrod which is at least partially disposed along the sleeve element.

According to some embodiments, the cam includes one or more lobes (e.g. rounded projections). Each of the one or more lobes is configured to engage a proximal end of the pushrod when the cam effects rotary motion. According to some such embodiments, each of the one or more lobes may distally projects from a distal end of the cam, such as to facilitate engaging the pushrod.

According to some embodiments, a distal end of the cam defines an oblique surface (i.e. angled relative to a lateral cross-section of the bent section), the cam being thereby configured to engage a proximal end of the pushrod when the cam effects rotary motion.

According to some embodiments, the pushrod extends in parallel, or substantially in parallel, to a central axis of the distal section (e.g. a rotational symmetry axis of the distal section when the distal section has a circular cross-section and is symmetric, or substantially symmetric, under said rotations). The pushrod may be displaced relative to the central axis of the distal section, thereby facilitating translation of the cam rotary motion into the axial, reciprocating motion of the pushrod.

According to some embodiments, the angle between the main section and the distal section is smaller than about 160°.

According to some such embodiments, the angle between the main section and the distal section is at least 90°.

According to some embodiments, the surgical tool is configured to allow operation thereof when the cable is bent at a bend radius lower than about 15 mm.

According to some embodiments, the surgical tool is configured to allow operation thereof when the cable is bent at a bend radius lower than about 12 mm.

According to some embodiments, the surgical tool is configured to allow operation thereof when the cable is bent at a bend radius lower than about 10 mm.

According to some embodiments, the surgical tool is configured to allow operation thereof when the cable is bent at a bend radius lower than about 9 mm.

According to some embodiments, the surgical tool is configured to allow operation thereof when the cable is bent at a bend radius lower than about 5 mm.

According to some embodiments, the surgical tool is configured to allow operation thereof when the cable is bent at a bend radius lower than about 2 mm.

According to some embodiments, the surgical is configured to allow the cable to generate rotary motion at rates of about 10,000-120,000 RPM, 20.000-120,000 RPM, 40,000-120,000 RPM, or any other range within 10,000-120,000 RPM.

According to some embodiments, the surgical tool is configured to generate axial, reciprocating motion of the headpiece such as to allow hammering of hard tissue at rates of about 10,000-480,000 SPM, 20,000-480,000 SPM, or any other range within 10,000-480,000 SPM. More generally, according to some embodiments, the SPM may equal the RPM times n, wherein n is the number of lobes on the cam.

According to some embodiments, the cable includes a plurality of wires. According to some such embodiments, the wires are braided/intertwined/stranded (for example, as described with respect to cable 130).

According to some embodiments, the surgical tool further includes a handle configured to facilitate operation and control of the surgical tool by an operator (e.g. a surgeon).

According to some such embodiments, the conversion of the rotary motion of the cable into axial, reciprocating motion of the headpiece is generated distally to the handle.

According to some embodiments, the hollow member includes at least two lumens. The cable is disposed along a first lumen (from the at least two lumens). A second lumen (from the at least two lumens) is connected to an operational input (e.g. an electrical port, a port for introducing fluid) and may be configured for the introduction thereinto of e.g. sensing instruments (such as a camera), fluid for irrigating the target tissue site.

According to some embodiments, a distal tip of the headpiece includes an eroding surface configured for hammering hard tissue. According to some such embodiments, the eroding surface includes one or more protrusions configured to facilitate the breaking up of the hard tissue. According to some such embodiments, the protrusions may be formed by diamonds embedded on the eroding surface.

According to some embodiments, a circumferential surface of the headpiece is eroding, the surgical tool being thereby configured for debulking hard tissue by grating (in addition to, or alternatively to, debulking hard tissue by hammering, as described above).

According to some embodiments, the stopper mechanism is at least partially spring-based.

According to an aspect of some embodiments, there is provided a method for debulking hard tissue. The method includes providing a surgical tool, such as surgical tool 100, surgical tool 200, or a surgical tool similar thereto, as described above. Guiding the distal section of the surgical tool to a target site in a vicinity the hard tissue intended for removal. Positioning the headpiece of the surgical tool in proximity to the hard tissue such as to allow hammering thereof Effecting axial, reciprocating motion of the headpiece such as to hammer and break up/fragment the hard tissue.

According to some embodiments, the headpiece of the surgical tool may be positioned adjacently to a surface of the hard tissue such as to allow grating thereof. In such embodiments, in the effecting of the axial, reciprocating motion of the headpiece, the headpiece fragments the hard tissue by grating the surface thereof.

According to an aspect of some embodiments, there is provided a surgical tool, such as surgical tool 300 or similar thereto, for breaking up/fragmenting hard tissue (e.g. bone tissue). The surgical tool includes a hollow member, a cable, a work element (e.g. a plate or plate-like element configured for hammering hard tissue), a rotation actuator, and a motion converter. The hollow member is elongated and includes a main section and a distal section. The distal section may be positioned at an angle relative to the main section (such as to facilitate accessing difficult-to-reach anatomical sites, e.g. between vertebrae). The cable is elongated and includes a cable proximal end and a cable distal end. The cable extends within the main section there along and is configured to resist helixing. The work element is exposed on a sidewall of the distal section and is configured for transverse, reciprocating motion, wherein the work element radially projects from the sidewall. The rotation actuator is coupled to the cable proximal end and is configured to rotate the cable about a longitudinal axis of the cable. The motion converter is coupled to both the cable distal end and to the work element. The motion converter includes a rotatable cam and a spring. The work element includes an eroding surface configured for hammering hard tissue and coupled to the spring. The cam includes one or more projections configured to (directly or indirectly) couple to the work element as the cam revolves, such as to laterally push the work element. The spring is configured to exert a return force on the work element when the work element projects from the sidewall. The work element is thereby configured to generate the transverse, reciprocating motion when the cam revolves.

According to some embodiments, the spring is a leaf spring.

According to some embodiments, the angle between the main section and the distal section is smaller than about 180°.

According to some embodiments, the angle between the main section and the distal section is smaller than about 165°.

According to some such embodiments, the angle between the main section and the distal section is at least 90°.

According to some embodiments, the surgical is configured to allow the cable to generate rotary motion at rates of about 10,000-120,000 RPM, 20.000-120,000 RPM, 40,000-120,000 RPM, or any other range within 10,000-120,000 RPM.

According to some embodiments, the surgical tool is configured to generate transverse, reciprocating motion of the work element such as to allow hammering of hard tissue at rates of about 10,000-480,000 SPM, 20,000-480,000 SPM, or any other range within 10,000-480,000 SPM. More generally, according to some embodiments, the SPM may equal the RPM times n, wherein n is the number of projections (e.g. lobes) on the cam.

According to an aspect of some embodiments, there is provided a method for debulking hard tissue. The method includes providing a surgical tool, such as surgical tool 300, or a surgical tool similar thereto, as described above. Guiding the distal section of the surgical tool to a target site in a vicinity the hard tissue intended for removal. Positioning the work element of the surgical tool in proximity to the hard tissue such as to allow hammering thereof. Effecting transverse, reciprocating motion of the work element such as to hammer and break up/fragment the hard tissue.

As used herein, according to some embodiments, the term “set” may refer to a collection of elements but also to a single element. Thus, for example, a “set of objects” may refer to one or more objects (e.g. springs).

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination or as suitable in any other described embodiment of the disclosure. No feature described in the context of an embodiment is to be considered an essential feature of that embodiment, unless explicitly specified as such.

Although steps of methods according to some embodiments may be described in a specific sequence, methods of the disclosure may include some or all of the described steps carried out in a different order. A method of the disclosure may include a few of the steps described or all of the steps described. No particular step in a disclosed method is to be considered an essential step of that method, unless explicitly specified as such.

Although the disclosure is described in conjunction with specific embodiments thereof, it is evident that numerous alternatives, modifications and variations that are apparent to those skilled in the art may exist. Accordingly, the disclosure embraces all such alternatives, modifications and variations that fall within the scope of the appended claims. It is to be understood that the disclosure is not necessarily limited in its application to the details of construction and the arrangement of the components and/or methods set forth herein. Other embodiments may be practiced, and an embodiment may be carried out in various ways.

The phraseology and terminology employed herein are for descriptive purpose and should not be regarded as limiting. Citation or identification of any reference in this application shall not be construed as an admission that such reference is available as prior art to the disclosure. Section headings are used herein to ease understanding of the specification and should not be construed as necessarily limiting.

1.-53. (canceled)

54. A surgical tool for debulking hard tissue, the surgical tool comprising:
a hollow member, which is elongated and comprises a distally located bent section;
a cable extending within the hollow member, along a predetermined length thereof, from a cable proximal end to a cable distal end, the cable being configured to resist helixing;
a headpiece positioned at, or distally to, the bent section;
a rotation actuator coupled to the cable proximal end and configured to rotate the cable about a longitudinal axis thereof; and
a motion converter coupled to the cable distal end and to the headpiece, at least a part of the motion converter is positioned in, and/or distally, to the bent section, the motion converter being configured to transform rotational motion of the cable into an axial, reciprocating motion of the headpiece;
wherein the headpiece is configured to break up hard tissue by hammering thereof, when effecting axial, reciprocating motion, while simultaneously minimizing damage to soft tissue if struck.

55. The surgical tool of claim 54, wherein the motion converter comprises a cam, coupled to the cable distal end, and a pushrod mechanically coupled to, or comprising, the headpiece and configured to engage the cam and to effect axial, reciprocating motion.

56. The surgical tool of claim 55, further comprising a main section and a distal section, wherein the bent section is joined on a proximal end thereof to the main section and on a distal end thereof to the distal section, and wherein the cam is positioned in the distal section and wherein, at least during a part of the axial, reciprocating motion, the headpiece distally projects from a distal end of the distal section.

57. The surgical tool of claim 55, wherein the cam comprises one or more lobes, projecting from a distal end of the cam, such as to engage a proximal end of the pushrod when the cam effects rotary motion.

58. The surgical tool of claim 55, wherein a distal end of the cam defines an oblique surface, the cam being thereby configured to engage a proximal end of the pushrod when the cam effects rotary motion.

59. The surgical tool of claim 54, wherein at least a portion of the cable, which extends along the bent section, is flexible.

60. The surgical tool of claim 54, further comprising a stopper mechanism configured to restrict axial displacement of the pushrod in at least the distal direction.

61. The surgical tool of claim 60, wherein the pushrod comprises a pinhole extending from one side-surface thereof to an opposite side-surface thereof, and wherein the stopper mechanism comprises a pin extending through the pinhole from one sidewall of the distal section to an opposite sidewall thereof, the pinhole having a diameter greater than the pin, thereby restricting axial displacement of the pushrod while allowing for axial reciprocating motion of the pushrod.

62. The surgical tool of claim 61, further comprising a linear-motion bearing, positioned at the distal end of the distal section such that the pushrod extends therethrough, the linear-motion bearing being configured to facilitate axial, reciprocating motion of the pushrod.

63. The surgical tool of claim 54, wherein the motion converter comprises a sleeve element mountable on or insertable into the distal section, the sleeve element comprising the pushrod which is at least partially disposed within and along the sleeve element and wherein the pushrod comprises a pinhole extending from one side-surface thereof to an opposite side-surface thereof, and wherein the stopper mechanism comprises a pin extending through the pinhole from one sidewall of the sleeve element to an opposite sidewall thereof, the pinhole having a diameter greater than the pin, thereby restricting axial displacement of the pushrod while allowing for axial reciprocating motion of the pushrod.

64. The surgical tool of claim 54, wherein the cable comprises a plurality of braided/intertwined/stranded wires.

65. The surgical tool of claim 54, wherein a distal tip of the headpiece comprises an eroding surface, comprising one or more protrusions, and configured for hammering hard tissue.

66. A surgical tool for debulking hard tissue, the surgical tool comprising:
a hollow member, which is elongated and comprises a main section and a distal section, the distal section being positioned at an angle relative to the main section;
a cable, which is elongated and comprises a cable proximal end and a cable distal end, the cable extending within the main section there along and being configured to resist helixing;
a work element exposed on a sidewall of the distal section and configured for transverse, reciprocating motion, wherein, at least during a part of the transverse, reciprocating motion, the work element radially projects from the sidewall;
a rotation actuator coupled to the cable proximal end and configured to rotate the cable about a longitudinal axis thereof; and
a motion converter coupled to the cable distal end and to the work element, the motion converter being configured to transform rotational motion of the cable into the transverse, reciprocating motion of the work element;
wherein the work element comprises a radially facing eroding surface, the work element being configured to break up hard tissue by hammering thereof, when effecting transverse, reciprocating motion, while simultaneously minimizing damage to soft tissue if struck.

67. The surgical tool of claim 66, wherein the motion converter comprises a rotatable cam and a resilient member, wherein the work element is coupled to the resilient member, wherein the cam comprises one or more projections configured to laterally push the work element as the cam revolves, and wherein the resilient member is configured to exert a return force on the work element when the work element projects from the sidewall, the work element being thereby configured to effect the transverse, reciprocating motion when the cam revolves, wherein the one or more projections are configured to directly laterally push the work element as the cam revolves.

68. A surgical tool for debulking hard tissue, the surgical tool comprising:
an elongated hollow member comprising a main section and a distal section;
a cable extending within the hollow member along a predetermined length thereof, from a cable proximal end to a cable distal end, the cable being configured to resist helixing;
a headpiece positioned at the distal section;
a rotation actuator coupled to the cable proximal end and configured to rotate the cable about a longitudinal axis thereof; and
a motion converter positioned in, or in proximity to, the distal section, the motion converter being coupled to the cable distal end and to the headpiece, and configured to transform rotation of the cable into an axial or transverse, reciprocating motion of the headpiece;
wherein the headpiece is configured to break up hard tissue by hammering thereof, when effecting axial or transverse, reciprocating motion, while simultaneously minimizing damage to soft tissue if struck.

69. The surgical tool of claim 68, wherein the headpiece is positioned at the distal section of the hollow member, such as to be excentric.

70. The surgical tool of claim 68, further comprising one or more electrodes positioned on a distal tip of hollow member, such as to render the surgical tool configured for electrophysiological monitoring and/or neurostimulation.

71. The surgical tool of claim 70, further comprising one or more electrical wires embedded within a wall of the hollow member such that respective distal ends of the one or more wires are connected to the one or more electrodes respectively.

72. The surgical tool of claim 70, wherein at least part of the hollow member is made of an electrically conducting material(s) extending along a length of the hollow member until the distal tip thereof, wherein the distal tip constitutes the one or more electrodes and is configured to function as a single electrode, the hollow member being thereby configured to allow establishing a voltage between the one or more electrodes and an external electrode placed on/in a body of a subject during a hard tissue debulking procedure.

73. The surgical tool of claim 70, wherein at least part of the hollow member is made of an electrically conducting material(s) extending along a length of the hollow member until the distal tip thereof, wherein the one or more electrodes comprise at least two electrodes, wherein the distal tip constitutes at least two electrodes and is configured to function as two electrodes, the hollow member being thereby configured to allow establishing a voltage difference between the at least two electrodes.
